SEQUENCE LISTING (37 sequences)

SEQ ID NO: 1 - protein, 64 aa, Escherichia phage T7
  source 1..64: note = T7-phage Gp2 protein
MSNVNTGSLSVDNKKFWATVESSEHSFEVPIYAETLDEALELAEWQYVPAGFEVTRVRPCVAPK

SEQ ID NO: 2 - protein, 86 aa, Escherichia phage T7
  source 1..86: note = T7-phage Gp5.7 protein
GPEDTTQDDDMKQSIVKWLFELNAKQREVLARRFGLLGYEAATLEDVGREIGLTRERVRQIQVEGLRRLREILQTQGLNIEALFRE

SEQ ID NO: 3 - protein, 110 aa, Shamshuipovirus HK022
  source 1..110: note = phage HK022 Nun protein
MVKKTIYVNPDSGQNRKVSDRGLTSRDRRRIARWEKRIAYALKNGVTPGFNAIDDGPEYKINEDPMDKVDKALATPFPRDVEKIEDEKYEDVMHRVVNHAHQRNPNKKWS

SEQ ID NO: 4 - protein, 883 aa, Escherichia phage T7
  source 1..883: note = T7-phage Gp0.7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 5 - protein, 52 aa, Escherichia phage T7
  source 1..52: note = T7-phage Gp6 protein
MRKSLIMGTKEDVAKMKAKRQMNKAVTFAERYSTSEPVRRIVTFNHPAIKGM

SEQ ID NO: 6 - protein, 71 aa, Escherichia phage T7
  source 1..71: note = T7-phage Gp8 protein
MEQLNYGYKIKRNQVRGSWLFLVYGKPIYELHRGEKSKTYYVTHIATGKTPACAGLLRDAIMKACMLEGLL

SEQ ID NO: 7 - protein, 341 aa, unidentified
  source 1..341: note = A* phage protein
MKSRRGFAIQRLMNAMRQAHADGWFIVFDTLTLADDRLEAFYDNPNALRDYFRDIGRMVLAAEGRKANDSHADCYQYFCVPEYGTANGRLHFHAVHFMRTLPTGSVDPNFGRRVRNRRQLNSLQNTWPYGYSMPIAVRYTQDAFSRSGWLWPVDAKGEPLKATSYMAVGFYVAKYVNKKSDMDLAAKGLGAKEWNNSLKTKLSLLPKKLFRIRMSRNFGMKMLTMTNLSTECLIQLTKLGYDATPFNQILKQNAKREMRLRLGKVTVADVLAAQPVTTNLLKFMRASIKMIGVSNLQSFIASMTQKLTLSDISDESKNYLDKAGITTACLRIKSKWTAGGK

SEQ ID NO: 8 - protein, 69 aa, unidentified
  source 1..69: note = Bacillus subtilis phage YkzG protein
MIYKVFYQEKADEVPVREKTDSLYIEGVSERDVRTKLKEKKFNIEFITPVDGAFLEYEQQSENFKVLEL

SEQ ID NO: 9 - protein, 350 aa, Bacillus phage SPO1
  source 1..350: note = Bacillus phage SPO1 GP40 protein
MHIYTYWGLKYVPSNSTMVAKEGDLILLGNEVHKVVKVLHRFRNITDLQITNWKGTETRYNLHVTEYKVLVPYDTHKEENEAMSDSLITHNGKDYVLCKIPARVGDLIRTEDKRVWEVLQKSKDGLVLYNEEKGEQRSAVYSEIGPYHVLVPRDTDTHTPTREELAAVIMNKAFTRTETQDSQEDTGTHKGLGLTGTDLYHSLRDLDAKVQSGYYTATENEEDVKSEIEATKKHMKAVKESGKTVNDYRKEENTKRCKLKALTNKFNRLFLKSVIDTDSLQVGKAYLIGGRDMKNVHGLYTGTTFDQQHANFLIVETDRMHRTLTVSAEQLFAEERHIVDIEKRVEQTED

SEQ ID NO: 10 - protein, 202 aa, Staphylococcus phage G1
  source 1..202: note = Staphylococcus phage G1 GP67 protein
MTNSKKKGDTFERKIAKELTAWWGYQFNRSPQSGGASWGKDNNAVGDIVVPQEANFPLVVECKHREEWTIDNVLLNNREPHTWWEQVINDSSKVNKTPCLIFTRNRAQSYVALPYDEKVYEDLRNNEYPVMRTDFIIDNIRKDKFFYDVLITTMNGLTSFTPSYIISCYDKKDIKPYKKVESNLSEVSKHEDELINDLLSDI

SEQ ID NO: 11 - protein, 141 aa, Thermus phage P23-45
  source 1..141: note = Thermus thermophilus phage P23-45 GP39 protein
MVEGFVEPYIRLFEAIPDAETELATFYDADLDTLPPRMFLPSGDLYTPPGPVRLEEIKRKRRVRLVKVSIYRFEHVGLGLAARPYAYAYAWQGDNGILHLYHAPVVLEDVPEVLELDEVTYNESYVRLMRAMGHVDAFIDL

SEQ ID NO: 12 - protein, 82 aa, Escherichia phage phiEco32
  source 1..82: note = Enterobacteria phage PhiEco32 GP79
MDMFSLEDLVQNGMMEQKEPLIVGSRKELRKLCEEWGITNQRMIGNQFSAIVTFLKRGDKYSMECVERIITEAQQDKGVTYL

SEQ ID NO: 13 - protein, 73 aa, Xanthomonas phage Xp10
  source 1..73: note = Xanthomonas oryzae phage Xp10 P7 protein
MNEFTQISGYVNAFGSQRGSVLTVKVENDEGWTLVEEDFDRADYGSDPEFVAEVSSYLKRNGGIKDLTKVLTR

SEQ ID NO: 14 - protein, 167 aa, Escherichia phage T4
  source 1..167: note = Enterobacteria phage T4 Alc protein
MDLQLITTEMVVEAYGDTTDGISVFKGNRRVGYITGLKKDLAKQVKRKTTIKEYRNRRLEQARDMLPDAVEEMKVFLENQLAKYDCEVFINQTQPNVHINSCKCYIIVNPLTGKHRLGISNPNRSASDMAEDVEACFKISKSPAEHHILINGLSQDDIVEVIKTLCM

SEQ ID NO: 15 - protein, 90 aa, Escherichia phage T4
  source 1..90: note = Enterobacteria phage T4 Asia protein
MNKNIDTVREIITVASILIKFSREDIVENRANFIAFLNEIGVTHEGRKLNQNSFRKIVSELTQEDKKTLIDEFNEGFEGVYRYLEMYTNK

SEQ ID NO: 16 - RNA, 115 nt, synthetic construct
  source 1..115: mol_type = other RNA; note = RNA-OUT sequence / artificial
tcgcacatcttgttgtctgattattgatttttcgcgaaaccatttgatcatatgacaagatgtgtatccaccttaacttaatgatttttaccaaaatcattaggggattcatcag

SEQ ID NO: 17 - RNA, 27 nt, synthetic construct
  source 1..27: mol_type = other RNA; note = RNA-IN sequence / artificial
gcgaaaaatcaataatcagacaacaag

SEQ ID NO: 18 - RNA, 9147 nt, synthetic construct
  source 1..9147: mol_type = other RNA; note = IS10 sequence / artificial
ctgatgaatcccctaatgatttttatcaaaatcattaagttaaggtagatacacatcttgtcatatgatcaaatggtttcgccaaaaatcaataatcagacaacaaaatgtgcgaactcgatattttacacgactctctttaccaattctgccccgaattacacttaaaacgactcaacagcttaacgttggcttgccacgccttacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatatccgtgagcaaaaacggcttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatggattctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagactcactcgtggctgcaaccctgcttactcaaaatctattcacacatggttacgttttggggaaattatgaggggatctctcagtgctcagcatgtttgagtgctttactcgccgcgggttgagaaatatgtaaaacttcagccgcacgcgttaaggaaccacaagtcattacagcataaaagatatcgagatgtcttaatttcattccatgtagcctactgattatttatttctacggactattctaacgaataaagtataaataatcagatatactcgtcataattcaaattttggtttagttgtcacgagaattaattcgtaaacgtgcaagctaaatagattgactaggggaatatacggggtaagaaagcggctcggcatactgccctataataaattgttaggagaagtacgccgagtagtgtgtagctattaggtaaagtatgtaccaatttcaataaatattttaattaagatactgtttgaaatatcaacaataaaggcaccgaccataggaacgacaaggaaagctttatgtgatggtccaaatgcttttgtgactgtttgcatattcgcaattgctgttggtgttgctcccataccaaagccacagtgacccgcgctgatcacgacagcatcataatctttgcccatcattttgaaggtgacaaagcaggcaaatagcaccatgacaacagtttgtacagcaatgataattaatactggccctgccatgcttgccaattgaccaaattttaatgacattaacgccattgccaagaaaagcgataaagcaacgctacctaatacatcgacggtcggctcaaacacttcgtgtttaaatacatgagtcagtgtattacggataataataccgacaaataaacaccagacaaaagtaggcagttgcagaaaagtatctttaaacaatgcactgatatagccaccaacaacaatacagataatcagcattgaaatggtttcaataacgttatttgcattgatttttcttttgacgcttggttgctcaaaagcttcaacgatagtgtcgcgctcttgctcggttgttttaggaatagagacctttttcaaaagatgacgggcaacagggccgccaactaaaccacccaacactaatccaagtgttgcacaagccatcgctaattcaacggcgcctgttacaccatatttatcagcgagaatagggccccatgctccggcattaccatgaccacctgttagagtaattgaacctgcaattaagccaataaatggactttcattcatcatgacagccatactcatgccgacagtattttgaatggcgattaggatcgttactgcaatagttaatagaactaacggctttcctcctttaatcagtcgagaaaagtcagaacttagcccgatagaggaaaagaatgtgagcattaataaactttgcaatgaagcatcaaaagtaaacgaataacctgatgttttatcaataattaacagaacaattgcaacaataaagccaccaacgacggcttctggtatgtggtttttttgtaggaacggggtaaattttacgacaaacattcctatgagtagcgcgatacatgcgactaataatgtataactggcatctaggatcatttttttacctctattagtttaccagaattttcactataacaatttaacaatagtgaaaaatgaagttactttggttaactaatctttaacaatgagttttattagggtatccataacttttagttaatagggtcataattttagtgtgatcttaatcatgaaatattaaaaagattaaaataacaacaatggtatttaatgatgggtaaacctaaataccatgcgggatccgcactattttcggtgatagcaaaaaataaaaaagtataaataataaacaaaattataagagaaggagaacatgataagcgcaggccgagcaatttaaagaagtgtgatatacaacataaaacagcagcatgacctttctatatttcgttgcttatcgaagtgttaattttcgggtggattactctgtttgttgattaataacggagaaaaatatgattgctgtaatatttgaggtgcaaatacaacccgaccaacaaactcgctatttgactttagctgaggagttaagaccactattaagtcatgtagctggttttatttcaattgaacgttttcaaagtctagctacagaaggaaaaatgttatcgctatcttggtgggaaaacgaatacgcagttctgcaatggaaaaatcatgttttacatgcgaaagctcaacaagaagggcgagagtcaatatttgatttttacaaaattagtattgctcatattactcgcgaatattcatttaaaaaggacaaggataatgtttgatgttcacgttgttttagataatcaaataggacaattagcattactaggaaaaacattaggtaataaaggtattggattggaagggggagggatatttacggttggtgatgaatgccatgctcattttcttgttgaacaaggaaaggaagctaaaatagcgctagagcaagctggactgttagtacttgcgatccggacaccattaattcgtaagttaaaacaggaaaaaccgggggaacttggcgaaatagcacgagtattggcggagaataacattaatattttagtgcaatacagtgaccatgctaaccaactgatattaataacggacaatgatagtatggctgcatctgttacgctcccttgggcaataaagtgaacttgcgatggctaatttaatacgaaaagaggttacctttgagtcctcaatagccgcgataggggcggctatgtctgacatttcacgagttaaaatactcagtgctttgatggatgggcgagcttggacggccactgagctaagttctgtggcgaatatatcagcttcaacggcgagcagtcatttatctaaattattagattgccagctaatcacagtagtagctcaaggcaagcatcgttattttcggctagcaggaaaagatattgctgaattgatggaaagtatgatggggatctccttaaaccatggcgtacatgccaaagtttccacgccagtgcatttacgaaaagcacgtacttgctatgatcatttagctggcgaagttgccgttaagatctatgattccctttgtcaacagcaatggatcactgaaaatggttcaatgatcacattaagtggtattcaatattttcatgaaatgggaattgacgttccttccaaacattcacgtaaaatctgttgtgcgtgtttagattggagtgaacgccgtttccatttaggtgggtacgttggagccgcattattttcgctttatgaatctaaagggtggttaactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaatgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcggatttattgcagatagtagtgcatttgcctttttagcgtttatatctgaaggttggttagttttccctgttttaattttattggctggtggtgggatcgctttacctgcattacagggagtgatgtctatccaaacaaagagtcatcagcaaggtgctttacagggattattggtgagccttaccaatgcaaccggtgttattggcccattactgtttgctgttatttataatcattcactaccaatttgggatggctggatttggattattggtttagcgttttactgtattattatcctgctatcgatgaccttcatgttaacccctcaagctcaggggagtaaacaggagacaagtgcttagttatttcgtcaccaaatgatgttattccgcgaaatataatgaccctcttgataacccaagagggcattttttacgataaagaagatttagcttcaaataaaacctatctattttatttatctttcaagctcaataaaaagccgcggtaaatagcaataaattggccttttttatcggcaagctcttttaggtttttcgcatgtattgcgatatgcataaaccagccattgagtaagtttttaagcacatcatcatcataagctttaagttggttctcttggatcaatttgctgacaatggcgtttaccttaccagtaatgtattcaaggctaattttttcaagttcattccaaccaatgataggcatcacttcttggatagggataaggtttttattattatcaataatataatcaagataatgttcaaatatactttctaaggcagaccaaccatttgttaaatcagtttttgttgtgatgtaggcatcaatcataattaattgctgcttataacaggcactgagtaattgttttttatttttaaagtgatgataaaaggcacctttggtcaccaacgcttttcccgagatctcatctattgaaacagcttgatagcctttttcaacaaacaatattcgtgctgagttaaccagtgattgataggtactcttaaaattttcttgttgatgatttttattttccatgatagatttaaaataacataccgtcagtatgtttatggtatcatgatgatgtggtcgtgacaatcttaagaacatttaggttattttatgtatattgaacagcattctcgctatcaaaataaagctaataacatccaattaagatatgatgataagcagtttcatacaacggttatcaaagatgttctattatggattgaacataatttagatcagtctttactgcttgatgatgtggcgaataaagcgggttataccaagtggtattttcagcggctgttcaaaaaagtaacaggggtcacactggctagctatattcgtgctcgtcgtttgacgaaagcggctgttgagttgaggttgacgaaaaaaactatccttgagatcgcattaaaatatcaatttgattcccaacaatcttttacacgtcgatttaagtacatttttaaggttacaccaagttattatcggcgtaataaattatgggaattggaggcaatgcactgagagatcccctcataatttccccaaagcgtaaccatgtgtgaataaattttgagctagtagggttgcagccacgagtaagtcttcccttgttattgtgtagccagaatgccgcaaaacttccatgcctaagcgaactgttgagagtacgtttcgatttctgactgtgttagcctggaagtgcttgtcccaaccttgtttctgagcatgaacgcccgcaagccaacatgttagttgaagcatcagggcgattagcagcatgatatcaaaacgctctgagctgctcgttcggctatggcgtaggcctagtccgtaggcaggacttttcaagtctcggaaggtttcttcaatctgcattcgcttcgaatagatattaacaagttgtttgggtgttcgaatttcaacaggtaagttagttgctagaacccatggctcctttgccgacgctgagtagattttaggtgacgggtggtgacaatgagtccgtgtcgagcgctgattttttcggcctttagagcgagatttatacaatagaatttggcatgagattggattgcttttagtcagcctcttatagcctaaagtctttgagtgactagatgacatatcatgtaagttgctgataggtttccagttttccgctcctaggtctgcatattgtacttttcctcttactcgacttaaccagtaccaacccagcttctcaacggatttataccatggcactttaaagccagcatcactgacaatgagcggtgtggtgttactcggtagaatgctcgcaaggtcggctagaaattggtcatgagctttctttgaacattgctctgaaagcgggaacgctttctcataaagagtaacagaacgaccgtgtagtgcgactgaagctcgcaataccataagtcgtttttgctcacgaatatcagaccagtcaacaagtacaatgggcatcgtattgcccgaacagataaagctagcatgccaacggtatacagcgagtcgctctttgtggaggtgacgattacctaacaatcggtcgattcgtttgatgttatgttttgttctcgctttggttggcaggttacggccaagttcggtaagagtgagagttttacagtcaagtaatgcgtggcaagccaacgttaagctgttgagtcgttttaagtgtaattcggggcagaattggtaaagagagtcgtgtaaaatatcgagttcgcacatcttgttgtctgattattgatttttcgcgaaaccatttgatcatatgacaagatgtgtatccaccttaacttaatgatttttaccaaaatcattaggggattcatcag

SEQ ID NO: 19 - RNA, 151 nt, synthetic construct
  source 1..151: mol_type = other RNA; note = RNA-IN/RNA-OUT sequence with promoters
cagaattggtaaagagagtcgtgtaaaatatcgagttcgcacatcttgttgtctgattattgatttttcgcgaaaccatttgatcatatgacaagatgtgtatccaccttaacttaatgatttttaccaaaatcattaggggattcatcag

SEQ ID NO: 20 - RNA, 42 nt, synthetic construct
  source 1..42: mol_type = other RNA; note = RNA-IN with part of the coding sequence (CDS) of a transposase. CDS starts with ATG
gcgaaaaatcaataatcagacaacaagatgtgcgaactcgat

SEQ ID NO: 21 - RNA, 62 nt, synthetic construct
  source 1..62: mol_type = other RNA; note = RNA-IN / artificial
tagacacacatcttgtcatatgatagaatggtttcgccaaaaatcaataatcagacaacaag

SEQ ID NO: 22 - RNA, 27 nt, synthetic construct
  source 1..27: mol_type = other RNA; note = RNAIN / artificial
gccaaaaatcaataatcagacaacaag

SEQ ID NO: 23 - RNA, 147 nt, synthetic construct
  source 1..147: mol_type = other RNA; note = RNAOUT-wt / artificial
gtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtac

SEQ ID NO: 24 - RNA, 147 nt, synthetic construct
  source 1..147: mol_type = other RNA; note = RNAOUTv1 / artificial
gtggtagaattggtaaagagagtcgtgtaaaatatcgagttcgcacatcttgttgtctgattattgatttttggcgaaaccatttgatcatatgacaagatgtgtatctaccttaaaaaatcattagggatacttaatgatttttac

SEQ ID NO: 25 - RNA, 139 nt, synthetic construct
  source 1..139: mol_type = other RNA; note = RNAOUTv2 / artificial
gtagaattggtaaagagagtcgtgtaaaatatcgagttcatgacaagatgtgtctgattattgatttttggcgaaaccatttgatcatgcacatcttgttgtatctaccttaaaaaatcattagggatacttaatgatt

SEQ ID NO: 26 - DNA, 33 bp, synthetic construct
  source 1..33: mol_type = other DNA; note = Primer sequence / BsaI_pHSG_for
aggtctctctagaaccgtttttatcaggctctg

SEQ ID NO: 27 - DNA, 36 bp, synthetic construct
  source 1..36: mol_type = other DNA; note = Primer sequence / BsaI_pHSG_rev_V1
aggtctctggagtatttcttccagaattgccatgat

SEQ ID NO: 28 - DNA, 87 bp, synthetic construct
  source 1..87: mol_type = other DNA; note = Primer sequence / RNA-IN_murA_Int_FOR
attgtgacttcgccctggagcttcgttggcccctgaacacgaaatttatccatttagtttgttctcagttaagcggccgcgatgtgc

SEQ ID NO: 29 - DNA, 90 bp, synthetic construct
  source 1..90: mol_type = other DNA; note = Primer sequence / RNA-IN_ibaG_INT_rev
tcatgctgtgtcgatcaaagcgtatacccctgcggagtgggcgcgcgatcgcaaactgaacggcttttgaggctctggcagtgaatgggg

SEQ ID NO: 30 - DNA, 25 bp, synthetic construct
  source 1..25: mol_type = other DNA; note = Primer sequence / murA_RNA_seq_for
gccagtagtgcggcaaaaaggatag

SEQ ID NO: 31 - DNA, 24 bp, synthetic construct
  source 1..24: mol_type = other DNA; note = Primer sequence / Cat-seq_FOR
gctggtgatatgggatagtgttca

SEQ ID NO: 32 - DNA, 31 bp, synthetic construct
  source 1..31: mol_type = other DNA; note = Primer sequence / BbsI_pUC_rev
agaagactcgtacttaagccagccccgacac

SEQ ID NO: 33 - DNA, 31 bp, synthetic construct
  source 1..31: mol_type = other DNA; note = Primer sequence / BsaI_pUC_back
agaagactcgtcatcttccgcttcctcgctc

SEQ ID NO: 34 - DNA, 909 bp, synthetic construct
  source 1..909: mol_type = other DNA; note = Gene sequence / GFP_pL_Part1
aggtctctctccgagagcgttcaccgacaaacaacagataaaacgaaaggcccagtctttcgactgagcctttcgttttatttgatgcctggagatccttactcgagtttggatccttattatttatacagttcatccatgccatgcgtaatacccgccgcggtcacaaattccagcagcaccatgtgatcgcgtttttcgttcggatctttgctcagcgcgctctgggtgctcagataatggttatccggcagcagcaccgggccatcaccaatcggggtgttctgctgataatgatccgccagctgcacgctgccatcttcaatgttatggcgaattttaaagttcactttaataccgtttttctgtttatccgccataatatacacgttatggctgttatagttatattccagtttatggcccagaatgttaccatcttctttaaaatcaatgcctttcagttcaatgcggttcaccagggtatcaccttcaaatttcacttccgcgcgggttttatagttgccatcatctttaaaaaaaatggtgcgttcctgcacataaccttccggcatcgcgcttttaaaaaaatcatgctgtttcatgtgatccggatagcgcgcaaagcactgcacgccataaccaaaggtggtcaccagggtcggccacggcaccggcagtttgccggtggtgcaaataaatttcagggtcagtttaccataggtcgcatcgccttcaccttcgccgctcacgctaaatttatgaccgttcacatcgccatccagttccaccagaatcggcaccacaccggtaaacagttcttcgcctttgctcatatgtatatctccttcttaaagcggccgcgatgtgctcagtatctctatcactgatagggatgtcaagagacct

SEQ ID NO: 35 - DNA, 1021 bp, synthetic construct
  source 1..1021: mol_type = other DNA; note = Gene sequence / pL_RNA-IN-tetR_Part_2
aggtctctgtcaatctctatcactgatagggaaagcttaaaaggccatccgtcaggatggccttctgtcgactgcaggtagacacacatcttgtcatatgatagaatggtttcgccaaaaatcaataatcagacaacaagatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaggatccggctgctaacaaagcccgaaaggaagctgagttggctgctgccaccgctgagcaataactagcataaccccttggggcctctaaacgggtcttgaggggttttttgctcgacagccactggatatcgagctcgcttggactcctgttgatagatccagtaatgacctcagaactccatctggatttgttcagaacgctcggttgccgccgggcgttttttattggtgagaatccaagcactagagagacct

SEQ ID NO: 36 - DNA, 193 bp, synthetic construct
  source 1..193: mol_type = other DNA; note = gBLOCK / gBLOCK_RNAOUTv2
gcgctgcatcgatcgacgtaagaagacgtgtacgtaaaaatcattaagtatccctaatgattttttaaggtagatacacatcttgtcatatgatcaaatggtttcgccaaaaatcaataatcagacaacaagatgtgcgaactcgatattttacacgactctctttaccaattctaccacgtcacagtcttca

SEQ ID NO: 37 - DNA, 193 bp, synthetic construct
  source 1..193: mol_type = other DNA; note = gBLOCK / gBLOCK_RNAOUTv2
gcgctgcatcgatcgacgtaagaagacgtgtacgtaaaaatcattaagtatccctaatgattttttaaggtagatacaacaagatgtgcatgatcaaatggtttcgccaaaaatcaataatcagacacatcttgtcatgaactcgatattttacacgactctctttaccaattctaccacgtcacagtcttca

(19) United States
(12) Patent Application Publication
(10) Pub. No.: US 2026/0265763 A1
(43) Pub. Date: Sep. 10, 2026

(54) MEANS AND METHODS FOR ANTIBIOTIC-FREE PROPAGATION OF PLASMID DNA

(71) Applicant: ENGENES BIOTECH GMBH, Wien (AT)

(72) Inventor: Jürgen MAIRHOFER, Wien (AT)

(73) Assignee: ENGENES BIOTECH GMBH, Wien (AT)

(21) Appl. No.: 19/167,821

(22) PCT Filed: Mar. 22, 2024
(86) PCT No.: PCT/EP2024/057704

§ 371 Date: Sep. 22, 2025

(30) Foreign Application Priority Data
Mar. 23, 2023 (LU) 503714

(51) Int. Cl. C12N 15/70; C12N 15/63

(52) U.S. Cl. CPC C12N 15/70; C12N 15/635

(57) ABSTRACT

The present invention provides for means and methods for antibiotic-free propagation of plasmid DNA. Accordingly, host cells make use of the RNA-OUT/IN mechanism. Such host cells are used for the production of plasmid DNA. Plasmid DNA may contain a gene of interest which can be expressed in mammalian cells.

Plasmid DNA is being considered as a promising alternative to traditional protein vaccines or viral delivery methods for gene therapeutic applications. Traditionally, plasmid DNA is produced in bacterial cells using markers, such as antibiotic resistance genes, so as to force bacterial cells to keep and maintain plasmids. Accordingly, for selecting bacterial cells transformed with a plasmid and in order to assure maintenance of the plasmids in the bacterial cell, an antibiotic resistance gene is included in the plasmid backbone. Selection for plasmids is achieved by growing the cells in a medium containing the respective antibiotic, in which only plasmid bearing cells are able to grow.

However, mainly because of safety concerns, antibiotic resistance genes are not desired.

Accordingly, in order to overcome these safety concerns, efforts have been made to remove antibiotic resistance genes from the manufacture of plasmids and to develop alternative selection methods. For example, auxotrophic markers are used as selection principle, whereby the plasmid can compensate the auxotrophy of the bacterial cell.

Alternatively, the bacterial RNA I/II system or the RNA-IN/OUT was used as an antibiotic free plasmid selection system. Each of these systems has its own peculiarities and, thus, it cannot be expected that parts or elements of one system can be readily applied in the other system.

Indeed, The RNA-IN/OUT system is used exclusively with a direct counter-selectable marker (sacB) that allows plasmid selection on sucrose (Luke (2009), Vaccine 27, 6454-6459). In particular, RNA-OUT on a plasmid represses the expression of the chromosomally integrated, constitutively expressed sacB under the control of an RNA-IN promoter. In the presence of the plasmid, RNA-OUT represses sacB expression by pairing with RNA-IN, thereby preventing expression of sacB. In the absence of the plasmid, RNA-IN does not pair with RNA-OUT and sacB is expressed. Its gene product SacB, a levansucrase, converts sucrose into a toxic metabolite in Gram-negative bacteria. Luke et al, cited above, show that the use of a direct counter-selectable marker allows high-yield plasmid production, whereas the use of repressor/operator systems, which indirectly control a gene essential for growth, does not allow high fermentation yields.

The RNA I/II system is not used in the art with a direct counter-selectable marker gene because it has been found that the expression of such a counter-selectable marker gene cannot be completely switched off. Thus, basic levels of transcription and translation of the direct counter-selectable marker gene, e.g. sacB, will result in cell death or very poor growth (Mairhofer (2008), Biotechnol. J. 3, 83-89). The RNA I/II system is therefore used as follows: an operator sequence allows the expression of a chromosomal gene that is essential for growth. The expression of a repressor protein, which would bind to the operator and repress transcription of the chromosomal gene, is suppressed by RNA I/II pairing because the mRNA encoding the repressor protein is preceded by RNA II, which pairs with RNA I contained on a plasmid. In the absence of the plasmid, the repressor protein is expressed and binds to the operator, thereby repressing transcription of the chromosomal gene essential for growth.

Thus, while the RNA-IN/OUT system uses a (direct) counter-selectable marker gene to achieve high yields of a desired product, e.g. plasmids in fermentation, and to be independent of ColE1-type plasmids (which naturally express RNA I and RNA II), the RNA I/II system uses an (indirect) transcriptional repressor/operator approach, since a counter-selectable marker gene has been shown not to work with the RNA I/II system. In particular, Luke et al., cited above, teach that for the RNA IN/OUT system a direct counter-selectable marker is required for high-yield plasmid production and advise against indirect control of an essential gene via a repressor/operator element, as this does not provide high yields, whereas Mairhofer et al, teaches that a direct counter-selectable marker does not work well with the RNA I/II system and therefore teaches the use of a repressor/operator element, i.e. indirect control of a gene essential for growth.

In addition, both systems are self-contained and not interchangeable, i.e. each system is contextualised. Therefore, a pick and choose approach to exchange elements from the RNA IN/OUT system with elements from the RNA I/II system and vice versa is not expected to work automatically.

However, there is still a need for further methods for the production of plasmid DNA that lacks the antibiotic selection marker.

Accordingly, the technical problem underlying the present invention is to address this need.

The present invention solves the technical problem by providing means and methods for antibiotic-free propagation of plasmid DNA. These means and method are reflected in the claims and described herein.

Thus, the present invention provides a non-naturally occurring bacterial cell containing
(a) a nucleotide sequence
   (a1) encoding at least one repressor protein, the expression of which is to be regulated, and, operably linked thereto,
   (a2) a nucleotide sequence encoding parts of an RNA-IN sequence,
      (i) which is complementary to an RNA-OUT sequence that is transcribable from a plasmid containing said RNA-OUT sequence, and
      (ii) which is present upstream of the nucleotide sequence encoding said repressor protein together with a ribosome binding site which is upstream or downstream of parts of said RNA-IN sequence,
wherein the parts of said RNA-IN sequence are designed and positioned such that they guarantee sufficient RNA-RNA interaction of the complementary sequences, so that when the plasmid with an RNA-OUT sequence is present in said bacterial cell, the RNA-OUT transcribed from said plasmid binds to RNA-IN transcribed from said nucleotide sequence of (a) in an extent sufficient to inhibit translation of the mRNA encoding said at least one repressor protein.

The expression of the nucleotide sequence (a1) encoding said at least one repressor protein which inhibits growth of said bacterial cell is to be regulated. This regulation is achieved by the nucleotide sequence (a2) and, as far as applicable, by the presence or absence of a plasmid containing RNA-OUT. At least one repressor protein is preferably a direct repressor protein.

Preferably, nucleotide sequence (a2) is inserted between the ribosomal binding site and the start codon of said nucleotide sequence (a1).

Preferably, nucleotide sequence (a1) and said nucleotide sequence (a2) are linked such that they are transcribed as one RNA.

“RNA-OUT” is a non-coding RNA which is antisense to RNA-IN. RNA-OUT contains a stem-loop domain topped by a flexibly paired loop.

The term “RNA-OUT” when used herein encompasses the strand which is antisense to RNA-IN (herein referred to as “antisense RNA-OUT”) and also encompasses the strand which is sense to RNA-IN (herein referred to as “sense RNA-OUT”).

An exemplary RNA-OUT sequence is shown in SEQ ID NO: 16. A preferred RNA-OUT sequence is shown in SEQ ID NO: 23, 24, or 25.

“RNA-IN” is a non coding RNA which is sense to RNA-OUT. At its 5′-end RNA-IN is complementary to the top of the loop of RNA-OUT, and complementarity extends for about 30-35 nucleotides down one side of RNA-OUT.

The term “RNA-IN” when used herein encompasses the strand which is sense to RNA-OUT (herein referred to as “sense RNA-IN”) and also encompasses the strand which is antisense to RNA-OUT (herein referred to as “antisense RNA-IN”)

An exemplary RNA-IN sequence is shown in SEQ ID NO: 17 or SEQ ID NO: 20. A preferred RNA-IN sequence is shown in SEQ ID NO: 21 or 22.

RNA-OUT and RNA-IN were originally identified in the insertion sequence IS10 containing the transposase Tn10; see Kittle 1989 (DOI: 10.1016/0022-2836 (89) 90132-0). Transposition of IS10 is regulated by RNA-OUT and RNA-IN in that translation of IS10 transposase mRNA (RNA-IN) is inhibited by an IS10-encoded anti-sense RNA (RNA-OUT). Accordingly, RNA-OUT can pair with RNA-IN and vice versa.

IS10 thus comprises RNA-OUT and RNA-IN. IS10 is shown in SEQ ID NO: 18.

The usual pairing between RNA-OUT and RNA-IN is, when spoken in terms as defined herein, antisense RNA-OUT and sense RNA-IN. However, in accordance with the terms as defined herein, also sense RNA-OUT and antisense RNA-IN are capable of pairing. Such pairing between RNA-OUT and RNA-IN, e.g. antisense RNA-OUT and sense RNA-IN or sense RNA-OUT and antisense RNA-IN is also referred to herein as “RNA-OUT/RNA-IN mechanism”. An exemplary scheme showing RNA-OUT/RNA-IN pairing is shown in FIG. 1.

Accordingly, the RNA-OUT/RNA-IN mechanism as applied by the bacterial cells of the present invention encompasses both pairings, i.e., antisense RNA-OUT and sense RNA-IN or sense RNA-OUT and antisense RNA-IN. The antisense RNA-OUT and sense RNA-IN pairing is preferred.

When taken out its IS10 context, RNA-OUT and RNA-IN can be used to regulate, e.g. gene expression in a way as is applied in its natural context described above. The present invention makes use of the RNA-OUT/RNA-IN mechanism for regulating gene expression in a bacterial cell.

Preferably, the RNA-IN sequence contained by bacterial cell of the present invention and as described herein is preceded by a promoter. Such a promoter may preferably be constitutive or inducible.

RNA-IN and/or RNA-OUT as used herein may contain mutations.

RNA-OUT as described herein contains a stem-loop structure.

In the meaning of the present invention, the term “loop” preferably encompasses the unpaired loop structure of RNA-OUT. However, this term is not strictly limited to the mere loop region, but may also comprise the adjacent nucleotides of the stem region, preferably not more than two nucleotides. Typically, a loop contains 7 nucleotides.

RNA-OUT may contain mutations in either the stem structure or loop structure or both.

RNA-OUT may contain mutations in the stem structure. There may be 1, 2, 3, 4, 5, 6, 7,8, 9, 10 or more mutations in the stem-structure of RNA-OUT, as long as a stem structure is formed. The sequences forming the stem structure as shown in SEQ ID NO: 16 (RNA-OUT) SEQ ID NO: 23 (RNA-OUT) may serve as a basis for such mutations.

RNA-OUT may contain mutation(s) in the loop which may be a single nucleotide exchange or any number of nucleotide exchanges, e.g. 2, 3, 4, 5, 6 or 7 nucleotide exchanges, as long as a loop structure is formed. The sequences forming the loops shown in SEQ ID NO: 16 (RNA-OUT) or SEQ ID NO: 23 (RNA-OUT) may serve as a basis for such mutations.

RNA-OUT may contain mutations in either the stem structure or loop structure or both.

A modified RNA-OUT sequence is shown in SEQ ID NO: 24 or 25.

RNA-IN may contain mutations. There may be 1, 2, 3, 4, 5, 6, 7, 8, 9, 10 or more mutations in RTNA-IN, as long as it can still pair with RNA-OUT. The sequence shown in SEQ ID NO: 17 (RNA-IN) or SEQ ID NO: 20 (RNA-IN) or SEQ ID NO: 21 (RNA-IN) or SEQ ID NO: 22 (RNA-IN) may serve as a basis for such mutations.

RNA-IN and RNA-OUT, respectively, containing mutations as described above, may also be referred to as “mutated RNA-IN” or “mutated RNA-OUT” and are also encompassed by the term “RNA-IN” or “RNA-OUT”, respectively.

Functionality of mutated RNA-IN and/or RNA-OUT may be tested by in a bacterial cell of the present invention.

However, not only mutated RNA-IN or mutated RNA-OUT are encompassed by the term “RNA-IN” or “RNA-OUT”, respectively, but also “parts” of RNA-IN or RNA-OUT. Thus, the term “RNA-IN” also includes “parts” of an RNA-IN sequence. The term “RNA-OUT” also includes “parts” of an RNA-OUT sequence.

Functionality of parts of RNA-IN or RNA-OUT, respectively, may likewise be tested in a bacterial cell of the present invention.

A non-naturally occurring bacterial cell of the present invention preferably contains
SEQ ID NO: 21 (RNA-IN) and SEQ ID NO: 23 (RNA-OUT);
SEQ ID NO: 21 (RNA-IN) and SEQ ID NO: 24 (RNA-OUT);
SEQ ID NO: 21 (RNA-IN) and SEQ ID NO: 25 (RNA-OUT);
SEQ ID NO: 22 (RNA-IN) and SEQ ID NO: 23 (RNA-OUT);
SEQ ID NO: 22 (RNA-IN) and SEQ ID NO: 24 (RNA-OUT); or
SEQ ID NO: 22 (RNA-IN) and SEQ ID NO: 25 (RNA-OUT).

Preferably, an RNA-OUT as described herein pairs with an RNA-IN such that the ribosome binding site which is upstream or downstream of parts of said RNA-IN sequence is not accessible for ribosomes. As a result, translation cannot occur.

Preferably, RNA-OUT as described herein, while pairing with an RNA-IN as described herein, does not pair, preferably does not fully pair, with the start codon (ATG) of the nucleotide sequence encoding the repressor protein.

Accordingly, it is preferred that RNA-OUT as described herein pairs with an RNA-IN such that the ribosome binding site which is upstream or downstream of parts of said RNA-IN sequence is not accessible for ribosomes, but said RNA-OUT does not pair, preferably does not fully pair, with the start codon (ATG) of the nucleotide sequence encoding the repressor protein. An illustrative example of such an RNA-OUT of the present invention is SEQ ID NO: 25.

However, any other RNA-OUT can be readily designed to fulfill the requirement that RNA-OUT does not pair, preferably does not fully pair, with the start codon (ATG) of the nucleotide sequence encoding the repressor protein

Exclusion of the start codon of the nucleotide sequence encoding the repressor protein, is in contrast to the set-up provided by Luke et al., cited above. In fact, Luke et al. applies RNA-OUT sequences which pair with RNA-IN sequences such that a ribosome binding is not accessible for ribosomes and in addition RNA-OUT sequences pair with the start codon of the nucleotide sequence encoding the enzyme levansucrase (SacB) (see the RNA-OUT sequence within FIG. 3 of Luke et al.).

It is assumed that the inclusion of the start codon when RNA-OUT pairs with RNA-IN is required by Luke et al., cited above, in order to make sure that any mRNA encoding the direct counter-selectable marker, if transcription may despite RNA-IN and RNA-OUT pairing has occurred, is under no circumstances translated.

A promoter sequence as used herein is a non-coding expression control sequence preferably inserted nearby the start of the coding sequence of a nucleotide sequence and regulates its expression. Put into a simplistic yet basically correct way, it is the interplay of the promoter with various specialized proteins called transcription factors that determine whether or not a given nucleotide sequence may be transcribed and eventually translated into the actual protein encoded by the gene. It will be recognized by a person skilled in the art that any compatible promoter can be used for expression in bacterial cells. The promoter itself may be preceded by an upstream activating sequence, an enhancer sequence or combination thereof. These sequences are known in the art as being any nucleotide sequence exhibiting a strong transcriptional activity in a bacterial cell.

The term “inducible promoter” as used herein refers to a promoter that regulates the expression of an operably linked nucleotide sequence in response to the presence or absence of an endogenous or exogenous stimulus. Such stimuli can be but are not limited to chemical compounds or environmental signals. Examples of inducible promoters are lac or PBAD promoter.

Preferably, a bacterial cell of the present invention is transformed with a plasmid containing said RNA-OUT sequence. The RNA-OUT sequence may be preceded by a promoter. A suitable promoter may be the native RNA-OUT promoter, a variant thereof still capable of driving expression of RNA-OUT or any other promoter, e.g. an inducible promoter, as described herein.

SEQ ID NO: 19 shows RNA-IN/RNA-OUT sequence with promoters.

The term “transforming” as used herein means alteration of the genotype of a bacterial cell by introducing a nucleotide sequence, e.g. a plasmid. The nucleotide sequence with which a bacterial cell is transformed does not necessarily originate from a different source, but it will, at some point, have been external to the bacterial cell into which it is to be introduced.

Preferably, the plasmid with which a bacterial cell is transformed or which it contains, is a low, medium or high copy number plasmid, with high copy number plasmid being preferred.

Preferably, the plasmid with which a bacterial cell is transformed or which it contains has a size or more than 5 kb e.g. 6 kb, 7 kb, 8 kb, 9 kb, 10 kb or more, e.g. it has a size of 5-50 kb, 5-45 kb, 5-40 kb, 5-35 kb, 5-30 kb, 5-25 kb, 5-20 kb, 5-15 kb, 5-10 kb, 5-9 kb, 5-8 kb, 5-7 kb, 5-6 kb.

When referred to herein the term “at least one repressor protein” encompasses one repressor protein or more than one repressor protein, such as two, three, four, five or more repressor proteins, with one or two repressor proteins being preferred.

A “repressor protein” as in commonly known in molecular genetics including bacterial genetics and gene regulation, is a DNA- or RNA-binding protein that inhibits the expression of one or more genes by binding to the operator or associated silencers.

A repressor protein which inhibits growth of a bacterial cell of the present invention may preferably either directly or indirectly inhibit growth of a bacterial cell, i.e., a “direct repressor protein” or “indirect repressor protein”. An exemplary scheme, wherein a direct repressor protein is applied is shown in FIG. 2. An exemplary scheme, wherein an indirect repressor protein is applied is shown in FIG. 3.

It was observed that by making use of an indirect repressor protein, plasmid yields of more than 1000 mg/L were achieved. Thus, bacterial (host) cells or alternative bacterial (host) cells as described herein, which are equipped with the RNA-IN/OUT system and an indirect repressor protein achieve plasmid yields which pursuant to the teaching of the prior art; see Luke et al., cited above, could not have been expected.

In addition, the plasmid produced by the bacterial host cells of the present invention which are equipped with the RNA-IN/OUT system is of high quality, i.e., plasmid DNA is about 100% ccc DNA; see FIG. 8B.

The term “growth” of a bacterial host cell as used herein means an increase of cell number due to cell division. When “growth of a bacterial host cell” is inhibited as described herein, growth includes preferably transcription, DNA-replication and/or cell division.

“Direct inhibition” means that the repressor protein inhibits growth of said bacterial cell by directly interfering with a protein which is essential for growth of said bacterial cell.

Such direct interaction or interference may be by binding of the repressor protein to the protein which is essential for growth of said bacterial cell, thereby inhibiting growth of said bacterial cell. Preferably, inhibition of growth of said bacterial cell by a direct repressor protein is by inhibiting transcription, DNA-replication and/or cell division, with inhibition of transcription being preferred, and with inhibition through interaction or interference with bacterial RNA polymerase being more preferred.

A direct repressor protein may preferably be a phage protein, particularly one or more of the phage proteins described herein, which inhibits transcription, DNA-replication and/or cell division. Such phage proteins are, for example, Nun, Gp0.7, Gp6, Gp8 or A*, Bacillus subtilis YkzG protein, Bacillus phage SPO1 GP40, Staphylococcus phage G1 GP67, Thermus thermophilus phage P23-45 GP39, Enterobacteria phage PhiEco32 GP79, Xanthomonas oryzae bacteriophage Xp10 P7 protein, Enterobacteria phage T4 Alc protein, Enterobacteria phage T4 Asia.

The term “Nun” encompasses a protein which inhibits bacterial cell RNA polymerase, wherein said protein is preferably
(a) a protein having the amino acid sequence shown in SEQ ID NO: 3 or a fragment thereof which inhibits bacterial cell RNA polymerase; or
(b) a protein having an amino acid sequence which has an identity of 40% or more, such as 50%, 60%, 70%, 80% or 90% to the amino acid sequence shown in SEQ ID NO: 3 and which inhibits bacterial cell RNA polymerase.

The term “Gp0.7” encompasses a protein which inhibits bacterial cell RNA polymerase by phosphorylation, wherein said protein is preferably
(a) a protein having the amino acid sequence shown in SEQ ID NO: 4 or a fragment thereof which inhibits bacterial cell RNA polymerase by phosphorylation; or
(b) a protein having an amino acid sequence which has an identity of 40% or more, such as 50%, 60%, 70%, 80% or 90% to the amino acid sequence shown in SEQ ID NO: 4 and which inhibits bacterial cell RNA polymerase by phosphorylation.

The term “Gp6” encompasses a protein which inhibits bacterial cell DNA replication, wherein said protein is preferably
(a) a protein having the amino acid sequence shown in SEQ ID NO: 5 or a fragment thereof which inhibits bacterial cell DNA replication; or
(b) a protein having an amino acid sequence which has an identity of 40% or more, such as 50%, 60%, 70%, 80% or 90% to the amino acid sequence shown in SEQ ID NO: 5 and which inhibits bacterial cell DNA replication.

The term “Gp8” encompasses a protein which inhibits bacterial cell DNA replication, wherein said protein is preferably
(a) a protein having the amino acid sequence shown in SEQ ID NO: 6 or a fragment thereof which inhibits bacterial cell DNA replication; or
(b) a protein having an amino acid sequence which has an identity of 40% or more, such as 50%, 60%, 70%, 80% or 90% to the amino acid sequence shown in SEQ ID NO: 6 and which inhibits bacterial cell DNA replication.

The term “A*” encompasses a protein which inhibits bacterial cell DNA replication, wherein said protein is preferably
(a) a protein having the amino acid sequence shown in SEQ ID NO: 7 or a fragment thereof which inhibits bacterial cell DNA replication; or
(b) a protein having an amino acid sequence which has an identity of 40% or more, such as 50%, 60%, 70%, 80% or 90% to the amino acid sequence shown in SEQ ID NO: 7 and which inhibits bacterial cell DNA replication.

The term “YkzG*” encompasses a protein which inhibits bacterial cell RNA polymerase, wherein said protein is preferably
(a) a protein having the amino acid sequence shown in SEQ ID NO: 8 or a fragment thereof which inhibits bacterial cell RNA polymerase; or
(b) a protein having an amino acid sequence which has an identity of 40% or more, such as 50%, 60%, 70%, 80% or 90% to the amino acid sequence shown in SEQ ID NO: 8 and which inhibits bacterial cell RNA polymerase.

The term GP40″ encompasses a protein which causes bacterial cell transcription shut-off, wherein said protein is preferably
(a) a protein having the amino acid sequence shown in SEQ ID NO: 9 or a fragment thereof which causes bacterial cell transcription shut-off; or
(b) a protein having an amino acid sequence which has an identity of 40% or more, such as 50%, 60%, 70%, 80% or 90% to the amino acid sequence shown in SEQ ID NO: 9 and which causes bacterial cell transcription shut-off.

The term GP67″ encompasses a protein which causes bacterial cell transcription shut-off, wherein said protein is preferably
(a) a protein having the amino acid sequence shown in SEQ ID NO: 10 or a fragment thereof which causes bacterial cell transcription shut-off; or
(b) a protein having an amino acid sequence which has an identity of 40% or more, such as 50%, 60%, 70%, 80% or 90% to the amino acid sequence shown in SEQ ID NO: 10 and which causes bacterial cell transcription shut-off.

The term GP39″ encompasses a protein which causes bacterial cell transcription shut-off, wherein said protein is preferably
(a) a protein having the amino acid sequence shown in SEQ ID NO: 11 or a fragment thereof which causes bacterial cell transcription shut-off; or
(b) a protein having an amino acid sequence which has an identity of 40% or more, such as 50%, 60%, 70%, 80% or 90% to the amino acid sequence shown in SEQ ID NO: 11 and which causes bacterial cell transcription shut-off.

The term GP79″ encompasses a protein which causes bacterial cell transcription shut-off, wherein said protein is preferably
(a) a protein having the amino acid sequence shown in SEQ ID NO: 12 or a fragment thereof which causes bacterial cell transcription shut-off; or
(b) a protein having an amino acid sequence which has an identity of 40% or more, such as 50%, 60%, 70%, 80% or 90% to the amino acid sequence shown in SEQ ID NO: 12 and which causes bacterial cell transcription shut-off.

The term “P7” encompasses a protein which causes bacterial cell transcription shut-off, wherein said protein is preferably
(a) a protein having the amino acid sequence shown in SEQ ID NO: 13 or a fragment thereof which causes bacterial cell transcription shut-off; or
(b) a protein having an amino acid sequence which has an identity of 40% or more, such as 50%, 60%, 70%, 80% or 90% to the amino acid sequence shown in SEQ ID NO: 13 and which causes bacterial cell transcription shut-off.

The term “T4 Alc” encompasses a protein which causes bacterial cell transcription shut-off, wherein said protein is preferably
(a) a protein having the amino acid sequence shown in SEQ ID NO: 14 or a fragment thereof which causes bacterial cell transcription shut-off; or
(b) a protein having an amino acid sequence which has an identity of 40% or more, such as 50%, 60%, 70%, 80% or 90% to the amino acid sequence shown in SEQ ID NO: 14 and which causes bacterial cell transcription shut-off.

The term “T4 Asia” encompasses a protein which causes bacterial cell transcription shut-off, wherein said protein is preferably
(a) a protein having the amino acid sequence shown in SEQ ID NO: 15 or a fragment thereof which causes bacterial cell transcription shut-off; or
(b) a protein having an amino acid sequence which has an identity of 40% or more, such as 50%, 60%, 70%, 80% or 90% to the amino acid sequence shown in SEQ ID NO: 15 and which causes bacterial cell transcription shut-off.

A preferred direct repressor protein of the present invention which is encoded by a nucleotide sequence contained in a bacterial cell as described herein is T7-phage Gp5.7 protein or T7-phage Gp2 protein or both, i.e. T7-phage Gp5.7 and T7-phase Gp2 protein. Each of Gp2 and Gp5.7 inhibits transcription. Such inhibition of transcription is through interaction of each of Gp2 and Gp5.7 with bacterial RNA polymerase. Gp5.7 and Gp2 are described, e.g. in Tabib-Salazar 2017 (DOI: 10.1093/nar/gkx370).

The term “Gp2” encompasses a protein which inhibits bacterial host cell RNA polymerase, wherein said protein is preferably
(a) a protein having the amino acid sequence shown in SEQ ID NO: 1 or a fragment thereof which inhibits bacterial host cell RNA polymerase; or
(b) a protein having an amino acid sequence which has an identity of 40% or more, such as 50%, 60%, 70%, 80% or 90% to the amino acid sequence shown in SEQ ID NO: 1 and which inhibits bacterial host cell RNA polymerase.

The term “Gp5.7” encompasses a protein which inhibits bacterial host cell RNA polymerase, wherein said protein is preferably
(a) a protein having the amino acid sequence shown in SEQ ID NO: 2 or a fragment thereof which inhibits bacterial host cell RNA polymerase; or
(b) a protein having an amino acid sequence which has an identity of 40% or more, such as 50%, 60%, 70%, 80% or 90% to the amino acid sequence shown in SEQ ID NO: 2 and which inhibits bacterial host cell RNA polymerase

The latter option mentioned before, i.e. both T7-phage Gp5.7 and T7-phage Gp2 protein is a preferred “at least one repressor protein” encoded by a nucleotide sequence contained in a bacterial cell as described herein. The skilled person is aware how to express both Gp5.7 and Gp2, e.g. both genes are driven by one promoter, whereby both genes are separated by a spacer which contains a ribosome binding site. Such a construction may also be referred to as “translational fusion” between Gp5.7 and Gp2.

“Indirect inhibition” means that the repressor does not directly interact or directly interfere with a protein which is essential for growth of said bacterial cell but repress transcription of the gene encoding said protein which is essential for growth of said bacterial cell. Accordingly, such an indirect repressor protein inhibits growth of said bacterial cell by repressing transcription of a gene encoding a protein which is essential for growth of said bacterial cell.

A preferred indirect repressor protein of the present invention which is encoded by a nucleotide sequence contained in a bacterial cell as described herein is the Tet repressor.

In case of an indirect repressor protein as referred to herein, a bacterial host cell of the present invention preferably further contains
(b) a nucleotide sequence
   (b1) encoding a protein which is essential for growth of said bacterial cell, the expression of which is to be regulated, and, operably linked thereto,
   (b2) a nucleotide sequence comprising a promoter and operator which is recognized and bound by said repressor protein of (a);
wherein the parts of said RNA-IN sequence are designed and positioned such that they guarantee sufficient RNA-RNA interaction of the complementary sequences, so that when the plasmid with an RNA-OUT sequence is present in said bacterial cell, the RNA-OUT transcribed from said plasmid binds to RNA-IN transcribed from said nucleotide sequence of (a) in an extent sufficient to inhibit translation of the mRNA encoding said indirect repressor protein, thereby allowing expression of said protein of (b).

The expression of the nucleotide sequence (b1) encoding a protein which is essential for growth of said bacterial cell is to be regulated. This regulation is achieved by the nucleotide sequence (b2) and, as far as applicable, by the presence or absence of said repressor protein of (a) which is preferably an indirect repressor protein.

An “operator” when referred to herein is a nucleotide sequence which the (indirect) repressor protein recognizes and binds, whereby expression of the nucleotide sequence encoding a protein which is essential for growth of said bacterial cell is regulated. A preferred operator is the Tet operator. The Tet operator is recognized and bound by the herein preferred Tet repressor.

Preferably, nucleotide sequence (a) and (b) is integrated in the genome of said bacterial cell.

Preferably, nucleotide sequence (a) and (b) is a nucleotide sequence that is foreign to said bacterial cell.

In case of an indirect repressor protein which inhibits growth of said bacterial cell by repressing transcription of a gene encoding a protein which is essential for growth of said bacterial cell, such a protein which is essential for growth of said bacterial cell is preferably MurA.

In the alternative to the above described bacterial cells, also an alternative set-up of such bacterial host cells is possible. This means that RNA-OUT and RNA-IN may be changed in the set-up as described for nucleotide sequence (a) and (b).

Accordingly, in the alternative to the above described bacterial host cells, the present invention provides an alternative non-naturally occurring bacterial cell containing
(a) a nucleotide sequence
   (a1) encoding at least one repressor protein, the expression of which is to be regulated, and, operably linked thereto,
   (a2) a nucleotide sequence encoding parts of an RNA-OUT sequence,
      (i) which is complementary to an RNA-IN sequence that is transcribable from a plasmid containing said RNA-IN sequence, and
      (ii) which is present upstream of the nucleotide sequence encoding said repressor protein together with a ribosome binding site which is upstream or downstream of parts of said RNA-OUT sequence,
   wherein the parts of said RNA-OUT sequence are designed and positioned such that they guarantee sufficient RNA-RNA interaction of the complementary sequences, so that when the plasmid with an RNA-IN sequence is present in said bacterial cell, the RNA-IN transcribed from said plasmid binds to RNA-OUT transcribed from said nucleotide sequence of (a) in an extent sufficient to inhibit translation of the mRNA encoding said at least one repressor protein.

The expression of the nucleotide sequence (a1) encoding said at least one repressor protein which inhibits growth of said bacterial cell is to be regulated. This regulation is achieved by the nucleotide sequence (a2) and, as far as applicable, by the presence or absence of a plasmid containing RNA-IN. The at least one repressor protein is preferably a direct repressor protein.

Accordingly, in case of an indirect repressor protein as referred to herein, the alternative bacterial host cell (i.e., alternative non-naturally occurring bacterial cell) of the present invention preferably further contains
(b) a nucleotide sequence
   (b1) encoding a protein which is essential for growth of said bacterial cell, the expression of which is to be regulated, and, operably linked thereto,
   (b2) a nucleotide sequence comprising a promoter and operator which is recognized and bound by said repressor protein of (a);
   wherein the parts of said RNA-OUT sequence are designed and positioned such that they guarantee sufficient RNA-RNA interaction of the complementary sequences, so that when the plasmid with an RNA-IN sequence is present in said bacterial cell, the RNA-IN transcribed from said plasmid binds to RNA-OUT transcribed from said nucleotide sequence of (a) in an extent sufficient to inhibit translation of the mRNA encoding said indirect repressor protein, thereby allowing expression of said protein of (b).

The expression of the nucleotide sequence (b1) encoding a protein which is essential for growth of said bacterial cell is to be regulated. This regulation is achieved by the nucleotide sequence (b2) and, as far as applicable, by the presence or absence of said repressor protein of (a) which is preferably an indirect repressor protein.

Preferably, the RNA-OUT sequence contained by an alternative bacterial cell of the present invention and as described herein is preceded by a promoter. Such a promoter may preferably be constitutive or inducible.

Preferably, an alternative bacterial cell of the present invention is transformed with a plasmid containing said RNA-IN sequence.

An alternative non-naturally occurring bacterial cell of the present invention preferably contains
SEQ ID NO: 21 (RNA-IN) and SEQ ID NO: 23 (RNA-OUT);
SEQ ID NO: 21 (RNA-IN) and SEQ ID NO: 24 (RNA-OUT);
SEQ ID NO: 21 (RNA-IN) and SEQ ID NO: 25 (RNA-OUT);
SEQ ID NO: 22 (RNA-IN) and SEQ ID NO: 23 (RNA-OUT);
SEQ ID NO: 22 (RNA-IN) and SEQ ID NO: 24 (RNA-OUT); or
SEQ ID NO: 22 (RNA-IN) and SEQ ID NO: 25 (RNA-OUT).

Needless to say, that all embodiments, definitions, etc. as described herein for bacterial host cells and methods applying them, also apply to “alternative bacterial host cells” as described herein.

A bacterial cell (or sometimes referred herein as “bacterial host cell”) as described herein may be any bacterial cell, preferably a bacterial host cell is Escherichia coli, such as Escherichia coli K-12 JM108, Vibrio natriegens, Pseudomonas putida or Bacillus subtilis. The term “bacterial (host) cell” may be interchangeably used herein with the term “non-naturally occurring bacterial cell”.

The term “nucleotide sequence” or” nucleic acid molecule” as used herein refers to a polymeric form of nucleotides (i.e. polynucleotide) which are usually linked from one deoxyribose or ribose to another. The term “nucleotide sequence” preferably includes single and double stranded forms of DNA or RNA. A nucleic acid molecule of this invention may include both sense and antisense strands of RNA (containing ribonucleotides), cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

In this regard, a nucleic acid being an expression product is preferably a RNA, whereas a nucleic acid to be introduced into a cell is preferably DNA, e.g. genomic DNA or cDNA.

The nucleic acid can be in any topological conformation. For instance, the nucleic acid can be single-stranded, double-stranded, triple-stranded, quadruplexed, partially double-stranded, branched, hairpinned, circular, or in a padlocked conformation.

“Operably linked” expression control sequences refers to a linkage in which the expression control sequence is contiguous with the expression cassette, as well as expression control sequences that act in trans or at a distance to control expression of the expression cassette.

The term “expression” as used herein means the transcription of a nucleotide sequence. Said nucleotide sequence encodes preferably a protein. Accordingly, said term also includes the production of mRNA (as transcription product from a nucleotide sequence) and translation of this mRNA to produce the corresponding gene product, such as a polypeptide, or protein.

The terms “5′” and “3′” used herein refer to a convention used to describe features of a nucleotide sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, nucleotide sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left hand side, and 3′ (downstream) indicates genetic elements positioned towards the right hand side, when following this convention.

A “polypeptide” refers to a molecule comprising a polymer of amino acids linked together by peptide bonds. Said term is not meant herein to refer to a specific length of the molecule and is therefore herein interchangeably used with the term “protein”. When used herein, the term “polypeptide” or “protein” also includes a “polypeptide of interest” or “protein of interest” which is expressed by the expression cassettes or vectors or can be isolated from the host cells of the invention. A protein of interest also includes proteins which may potentially be harmful or even toxic for host cells.

The “polypeptide” as used herein encompasses both naturally-occurring and non-naturally-occurring proteins, and fragments, mutants, derivatives and analogs thereof. Polypeptides may be a polypeptide homologous (native) or heterologous to the host cell. The polypeptide of interest may also encompass a polypeptide native to the host cell, which is encoded by a nucleic acid sequence, which expression is controlled by one or more control sequences foreign to the nucleic acid sequence encoding the polypeptide. Polypeptides may be of any length. Polypeptides include proteins and/or peptides of any activity or bioactivity. A “peptide” encompasses analogs and mimetics that mimic structural and thus biological function.

Polypeptides may further form dimers, trimers and higher oligomers, i.e. consisting of more than one polypeptide molecule. Polypeptide molecules forming such dimers, trimers etc. may be identical or non-identical. The corresponding higher order structures are consequently termed homo- or heterodimers, homo- or heterotrimers etc.

The plasmid with which a bacterial host cell is transformed or which it contains, preferably further contains a gene of interest. Preferably, such gene of interest is operably linked with a eukaryotic promoter that allows expression in a mammalian cell.

The bacterial host cells of the present invention can be used for the production of plasmid DNA or for a protein of interest.

Accordingly, the present invention relates to a method for producing plasmid DNA, comprising the steps of
transforming a population of bacterial cells of the present invention with a plasmid containing an RNA-OUT sequence;
growing said population of bacterial host cell under conditions in which said repressor protein is expressible in the bacterial cells, whereby expression of said repressor protein completely or partially inhibits growth of plasmid-free bacterial cells such that plasmid-bearing bacterial cells outgrow plasmid-free cells; and
harvesting plasmid-bearing bacterial cells.

Said method for producing plasmid DNA may preferably, further comprise
isolating and purifying plasmid DNA from said harvested bacterial cell

Also, the present invention relates to a method for producing a protein of interest, comprising the steps of
transforming a population of bacterial cells of the present invention with a plasmid containing an RNA-OUT sequence and further containing a nucleotide sequence encoding a protein of interest under the control of a prokaryotic promoter that allows expression of said protein in said bacterial host cells;
growing said population of bacterial host cell under conditions in which said repressor protein is expressible in the bacterial cells, whereby expression of said repressor protein completely or partially inhibits growth of plasmid-free bacterial cells such that plasmid-bearing bacterial cells outgrow plasmid-free cells; and
harvesting the protein of interest from plasmid-bearing bacterial cells.

Said method for producing a protein of interest may preferably, further comprise
isolating and purifying isolating and purifying said protein of interest.

The alternative bacterial host cells of the present invention can also be used for the production of plasmid DNA or for a protein of interest.

Accordingly, the present invention relates to a method for producing plasmid DNA, comprising the steps of
transforming a population of alternative bacterial cells of the present invention with a plasmid containing an RNA-IN sequence;
growing said population of alternative bacterial host cell under conditions in which said repressor protein is expressible in the alternative bacterial cells, whereby expression of said repressor protein completely or partially inhibits growth of plasmid-free alternative bacterial cells such that plasmid-bearing alternative bacterial cells outgrow plasmid-free alternative bacterial cells; and
harvesting plasmid-bearing alternative bacterial cells.

Said method for producing plasmid DNA may preferably, further comprise
isolating and purifying plasmid DNA from said harvested alternative bacterial cell.

The above method for producing plasmid DNA is preferably run as a continuous production.

Also, the present invention relates to a method for producing a protein of interest, comprising the steps of
transforming a population of alternative bacterial cells of the present invention with a plasmid containing an RNA-IN sequence and further containing a nucleotide sequence encoding a protein of interest under the control of a prokaryotic promoter that allows expression of said protein in said alternative bacterial host cells;
growing said population of alternative bacterial host cell under conditions in which said repressor protein is expressible in the alternative bacterial cells, whereby expression of said repressor protein completely or partially inhibits growth of plasmid-free alternative bacterial cells such that plasmid-bearing alternative bacterial cells outgrow plasmid-free alternative bacterial cells; and
harvesting the protein of interest from plasmid-bearing alternative bacterial cells.

Said method for producing a protein of interest may preferably, further comprise isolating and purifying isolating and purifying said protein of interest.

Furthermore, the present invention provides for the use the bacterial cells described herein for producing plasmid DNA. In order to do so, said bacterial cells are beforehand transformed with a plasmid.

Moreover, the present invention provides for the use of the bacterial cells described herein for producing a protein of interest. Said protein of interest is contained by a plasmid with which said bacterial cells are beforehand transformed.

The present invention may also be characterized by the following items:

(1) A non-naturally occurring bacterial cell containing
(a) a nucleotide sequence
   (a1) encoding at least one repressor protein which inhibits growth of said bacterial cell, the expression of which is to be regulated, and, operably linked thereto,
   (a2) a nucleotide sequence encoding parts of an RNA-IN sequence,
      (i) which is complementary to an RNA-OUT sequence that is transcribable from a plasmid containing said RNA-OUT sequence, and
      (ii) which is present upstream of the nucleotide sequence encoding said repressor protein together with a ribosome binding site which is upstream or downstream of parts of said RNA-IN sequence,
wherein the parts of said RNA-IN sequence are designed and positioned such that they guarantee sufficient RNA-RNA interaction of the complementary sequences, so that when the plasmid with an RNA-OUT sequence is present in said bacterial cell, the RNA-OUT transcribed from said plasmid binds to RNA-IN transcribed from said nucleotide sequence of (a) in an extent sufficient to inhibit translation of the mRNA encoding said at least one repressor protein.

(2) The bacterial cell of any one of the preceding items, wherein said at least one repressor protein directly inhibits growth of said bacterial cell.

(3) The bacterial cell of any one of the preceding items, wherein said at least one repressor protein is T7-phage Gp5.7 protein and T7-phage Gp2 protein.

(4) The bacterial cell of item 1, wherein said at least one repressor protein which indirectly inhibits growth of said bacterial cell by repressing transcription of a gene encoding a protein which is essential for growth of said bacterial cell.

(5) The bacterial cell of item 1 or 4, wherein said at least one repressor protein is the Tet repressor.

(6) The bacterial cell of item 1, 4 or 5, further containing
(b) a nucleotide sequence
   (b1) encoding a protein which is essential for growth of said bacterial cell, the expression of which is to be regulated, and, operably linked thereto,
   (b2) a nucleotide sequence comprising a promoter and operator which is recognized and bound by said repressor protein of (a);
wherein the parts of said RNA-IN sequence are designed and positioned such that they guarantee sufficient RNA-RNA interaction of the complementary sequences, so that when the plasmid with an RNA-OUT sequence is present in said bacterial cell, the RNA-OUT transcribed from said plasmid binds to RNA-IN transcribed from said nucleotide sequence of (a) in an extent sufficient to inhibit translation of the mRNA encoding said indirect repressor protein, thereby allowing expression of said protein of (b).

(7) The bacterial cell of item 4 or 6, wherein said protein which is essential for growth of said bacterial cell is MurA.

(8) The bacterial cell of any one of the preceding items, wherein said nucleotide sequence (a) and (b) is integrated in the genome of said bacterial cell.

(9) The bacterial cell of any one of the preceding items, wherein said nucleotide sequence (a) and (b) is a nucleotide sequence that is foreign to said bacterial cell.

(10) The bacterial cell of any one of the preceding items, wherein said nucleotide sequence (a2) is inserted between the ribosomal binding site and the start codon of said nucleotide sequence (a1).

(11) The bacterial cell of any one of the preceding items, wherein said nucleotide sequence (a1) and said nucleotide sequence (a2) are linked such that they are transcribed as one RNA.

(12) The bacterial host cell of any one of the preceding items, wherein the RNA-IN sequence is preceded by a promoter.

(13) The bacterial host cell of item 12, wherein said promoter is constitutive or inducible.

(14) The bacterial cell of any one of the preceding items, wherein said bacterial cell is transformed with said plasmid containing said RNA-OUT sequence.

(15) The bacterial cell of item 14, wherein said plasmid further contains a gene of interest.

(16) The bacterial cell of item 15, wherein said gene of interest is operably linked with a eukaryotic promoter that allows expression in a mammalian cell.

(17) The bacterial cell of any one of items 14 to 16, wherein said plasmid is a high copy number plasmid.

(18) The bacterial cell of any one of items 14 to 17, wherein said plasmid has a size of more than 5 kb.

(19) The bacterial cell of any one of the preceding items, which is an Escherichia coli cell.

(20) A method for producing plasmid DNA, comprising the steps of
transforming a population of bacterial cells of any one of items 1 to 19 with a plasmid containing an RNA-OUT sequence;
growing said population of bacterial host cell under conditions in which said repressor protein is expressible in the bacterial cells, whereby expression of said repressor protein completely or partially inhibits growth of plasmid-free bacterial cells such that plasmid-bearing bacterial cells outgrow plasmid-free cells; and
harvesting plasmid-bearing bacterial cells.

(21) The method of item 20, further comprising
isolating and purifying plasmid DNA from said harvested bacterial cell.

(22) A method for producing a protein of interest, comprising the steps of
transforming a population of bacterial cells of any one of items 1 to 19 with a plasmid containing an RNA-OUT sequence and further containing a nucleotide sequence encoding a protein of interest under the control of a prokaryotic promoter that allows expression of said protein in said bacterial host cells;
growing said population of bacterial host cell under conditions in which said repressor protein is expressible in the bacterial cells, whereby expression of said repressor protein completely or partially inhibits growth of plasmid-free bacterial cells such that plasmid-bearing bacterial cells outgrow plasmid-free cells; and
harvesting the protein of interest from plasmid-bearing bacterial cells.

(23) The method of item 22, further comprising
isolating and purifying said protein of interest.

### FIGURES

FIG. 1 shows a schematic pairing of RNA-OUT and RNA-IN.

FIG. 2 shows an exemplary RNA-IN/OUT mechanism as described herein, whereby a direct repressor protein is applied. Gp2 and Gp5.7 are T7-phage proteins, RBS stands for ribosomal binding site, pBAD is an arabinose-inducible promoter.

FIG. 3 shows an exemplary RNA-IN/OUT mechanism as described herein, whereby an indirect repressor protein is applied. tetR stands for Tet repressor; tetO stands for Tet operator; murA encodes UDP-N-acetylglucosamine 1-carboxyvinyltransferase which is involved in murein biosynthesis.

FIG. 4 shows the in silico cloned pHSG567 harboring the TetO-RNOUT-TetR sequence.

FIG. 5 shows the in silico visualization of the PCR for amplification for the generation of the integration cassette. The grey highlighted section represents the PCR product.

FIG. 6 In silico visualization of the pUC19 based RNAOUT-plasmid.

FIG. 7 shows the result of the set-up shown in FIG. 3.

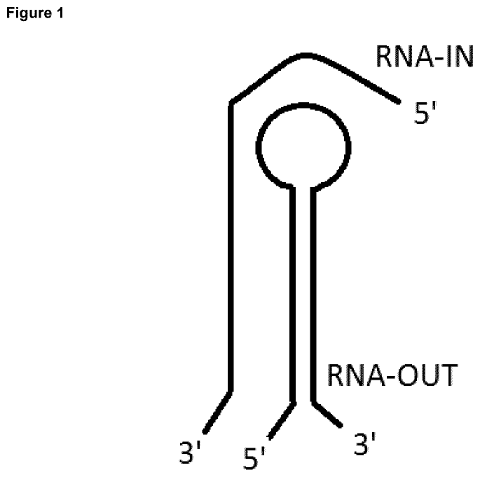

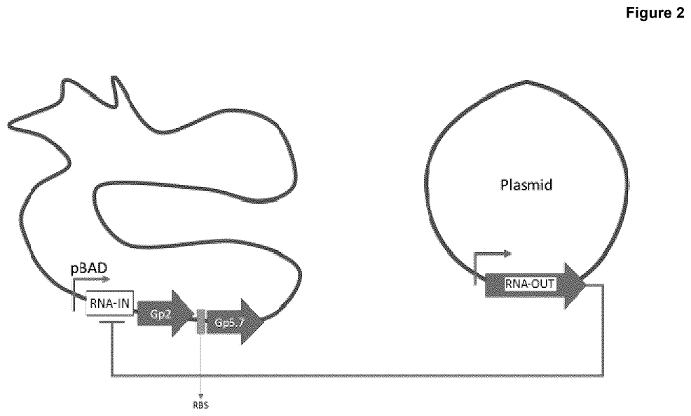

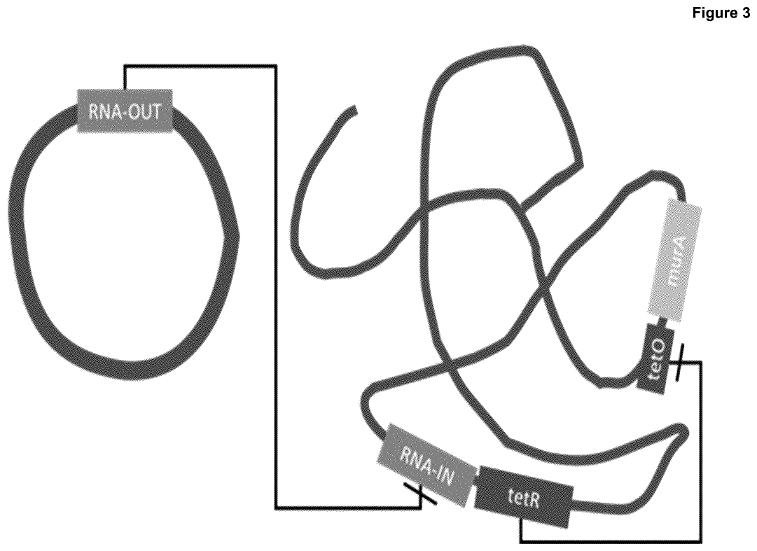

Upper left plate (A) shows growth of bacterial colonies which contain murA under the control of tetO and tetR operably linked with RNA-IN as well as a plasmid with RNA-OUT.

Upper right plate (B) shows no growth of bacterial colonies. It is a control, where bacterial cells which contain murA under the control of tetO and tetR operably linked with RNA-IN were transformed with pUC19 (not containing RNA-OUT).

Plate (C) on the bottom shows no growth of bacterial colonies. It is a further control, where bacterial cells which contain murA under the control of tetO and tetR operably linked with RNA-IN were transformed with water.

FIG. 8A to D show (A) productivity for closed covalent circular (ccc) plasmid DNA produced by bacterial host cells as described herein, (B) plasmid DNA (pDNA) quality produced by bacterial host cells as described herein, (C) volumetric yield of ccc DNA produced by bacterial host cells as described herein, (D) cell dry mass (CDM) produced by bacterial host cells as described herein.

Bacterial host cells were equipped with an RNA-IN sequence (SEQ ID NO: 22) and harbor pUC19 containing the RNA-OUT sequence (SEQ ID NO: 23), named “Plasmid_RNAOUT-wt” in FIGS. 8A to 8D.

Bacterial host cells were equipped with an RNA-IN sequence (SEQ ID NO: 22) and harbor pUC19 containing the modified RNA-OUT sequence (SEQ ID NO: 24), named “Plasmid_RNAOUTv1” in FIGS. 8A to 8D.

Bacterial host cells were equipped with an RNA-IN sequence (SEQ ID NO: 22) and harbor pUC19 containing the modified RNA-OUT sequence (SEQ ID NO: 25), named “Plasmid_RNAOUTv2” in FIGS. 8A to 8D.

### Sequences

SEQ ID NO: 1 shows T7-phage Gp2 protein
SEQ ID NO: 2 shows T7-phage Gp5.7 protein
SEQ ID NO: 3 shows phage HK022 Nun protein
SEQ ID NO: 4 shows T7-phage Gp0.7
SEQ ID NO: 5 shows T7-phage Gp6 protein
SEQ ID NO: 6 shows T7-phage Gp8 protein
SEQ ID NO: 7 shows A* phage protein
SEQ ID NO: 8 shows Bacillus subtilis phage YkzG protein
SEQ ID NO: 9 shows Bacillus phage SPO1 GP40 protein
SEQ ID NO: 10 shows Staphylococcus phage G1 GP67 protein
SEQ ID NO: 11 shows Thermus thermophilus phage P23-45 GP39 protein
SEQ ID NO: 12 shows Enterobacteria phage PhiEco32 GP79
SEQ ID NO: 13 shows Xanthomonas oryzae phage Xp10 P7 protein
SEQ ID NO: 14 shows Enterobacteria phage T4 Alc protein
SEQ ID NO: 15 shows Enterobacteria phage T4 Asia protein
SEQ ID NO: 16 shows RNA-OUT
SEQ ID NO: 17 shows RNA-IN
SEQ ID NO: 18 shows IS10
SEQ ID NO: 19 shows RNA-IN/RNA-OUT sequence with promoters
SEQ ID NO: 20 shows RNA-IN with part of the coding sequence (CDS) of a transposase. CDS starts with “ATG”
SEQ ID NO: 21 shows a RNA-IN sequence
SEQ ID NO: 22 shows a RNA-IN sequence
SEQ ID NO: 23 shows a RNA-OUT sequence, here a RNA-OUT wildtype sequence
SEQ ID NO: 24 shows a modified RNA-OUT sequence, i.e., RNA-OUTv1
SEQ ID NO: 25 shows a modified RNA-OUT sequence, i.e., RNA-OUTv2
SEQ ID NO: 26 shows a primer sequence
SEQ ID NO: 27 shows a primer sequence
SEQ ID NO: 28 shows a primer sequence
SEQ ID NO: 29 shows a primer sequence
SEQ ID NO: 30 shows a primer sequence
SEQ ID NO: 31 shows a primer sequence
SEQ ID NO: 32 shows a primer sequence
SEQ ID NO: 33 shows a primer sequence
SEQ ID NO: 34 shows the gene sequence of GFP (ordered gBLOCK)
SEQ ID NO: 35 shows the gene sequence of tetR (ordered gBLOCK)
SEQ ID NO: 36 shows the gene sequence of a modified RNA-OUT, i.e., RNA-OUTv2 (ordered gBLOCK)
SEQ ID NO: 37 shows the gene sequence of a modified RNA-OUT, i.e., RNA-OUTv2 (ordered gBLOCK)

It must be noted that as used herein, the singular forms “a”, “an”, and “the”, include plural references unless the context clearly indicates otherwise. Thus, for example, reference to “an expression cassette” includes one or more of the expression cassettes disclosed herein and reference to “the method” includes reference to equivalent steps and methods known to those of ordinary skill in the art that could be modified or substituted for the methods described herein.

All publications and patents cited in this disclosure are incorporated by reference in their entirety. To the extent the material incorporated by reference contradicts or is inconsistent with this specification, the specification will supersede any such material.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

The term “about” or “approximately” as used herein means within 20%, preferably within 10%, and more preferably within 5% of a given value or range. It includes also the concrete number, e.g., about 20 includes 20.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well-known in the art. Generally, nomenclatures used in connection with techniques of biochemistry, enzymology, molecular and cellular biology, microbiology, genetics and protein and nucleic acid chemistry and hybridization described herein are those well-known and commonly used in the art.

The methods and techniques of the present invention are generally performed according to conventional methods well-known in the art and as described in various general and more specific references that are cited and discussed throughout the present specification unless otherwise indicated. See, e. g., Sambrook et al., Molecular Cloning: A Laboratory Manual, 3rd ed., Cold Spring Harbor Laboratory Press, Cold Spring Harbor, N. Y. (2001); Ausubel et al., Current Protocols in Molecular Biology, J, Greene Publishing Associates (1992, and Supplements to 2002); Handbook of Biochemistry: Section A Proteins, Vol I 1976 CRC Press; Handbook of Biochemistry: Section A Proteins, Vol II 1976 CRC Press. The nomenclatures used in connection with, and the laboratory procedures and techniques of, molecular and cellular biology, protein biochemistry, enzymology and medicinal and pharmaceutical chemistry described herein are those well-known and commonly used in the art.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

### Example 1: RNA-IN/OUT System

The chosen strain for the implementation of this selection system is JM108. The aim was to integrate an RNA-IN like sequence upstream of Tet-Repressor protein (tetR) gene and to control an essential gene (murA) by modifying its promoter to contain Tet-Operator (tetO) sites to become controllable by said TetR protein. The first step to achieve this was to construct the described plasmid which later served as template for to generate dsDNA for the essential gene modification by homologous recombination (generating strain JM108_RAIN). Subsequently, overhang-primers were used to generate an integration cassette flanked by 50 bp overhangs homologous to the desired integration site, upstream of murA. After integration of tetO-RNAOUT-tetR cells would not be viable since the tetR suppresses the transcription of murA. Therefore, the antisense RNA-OUT sequence is necessary to stop the production of the TetR. An RNA-OUT sequence, e.g. RNA-OUTv2 was cloned into a pUC19 plasmid. If the JM108_RAIN needs to be cultivated without RNA-OUTv2-plasmid, anhydrotetracycline needs to be added, which inactivates TetR by a conformational change driving the repressors to dissociate from its complementary operators and allows the cells to grow.

### 1. Strain Engineering—JM108 RAIN

### 1.1 Cloning of the Plasmid for the Integration Cassette

For this task the plasmid of choice was the pHSG576 (pSC101 ori) [GenBank: D88215.1].

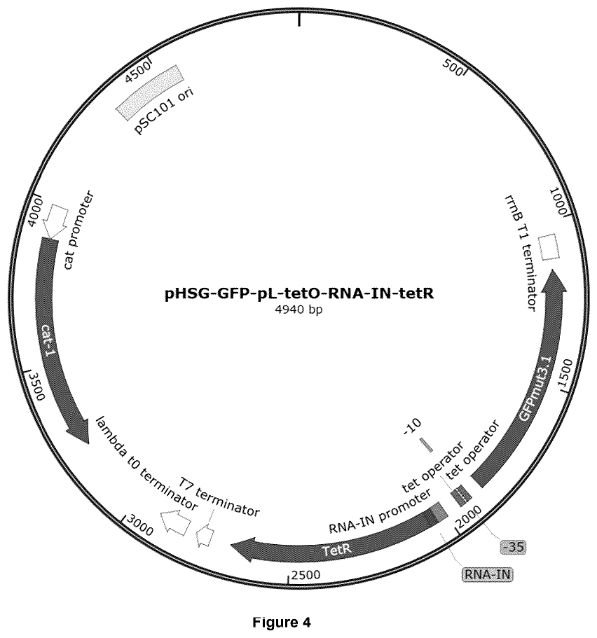

The primers Bsal_pHSG_for (SEQ ID NO: 26) and Bsal_pHSG_rev_V1 (SEQ ID NO: 27) were used for the amplification of the pHSG567 backbone. The PCR was carried out using the NEB Q5R Polymerase according to the provided instruction by NEB. Golden gate Assembly with Bsal was used to ligate the pHSG576 backbone with two inserts GFP_pL_Part1 (SEQ ID NO: 34) and pL_RNA-IN-tetR_Part_2 (SEQ ID NO: 35) provided by DNA synthesis. The result is shown in FIG. 4.

However, also the RNA-IN shown in SEQ ID NO: 17, 21 or 22 can be used.

### 1.2 Amplification of the Integration Cassette

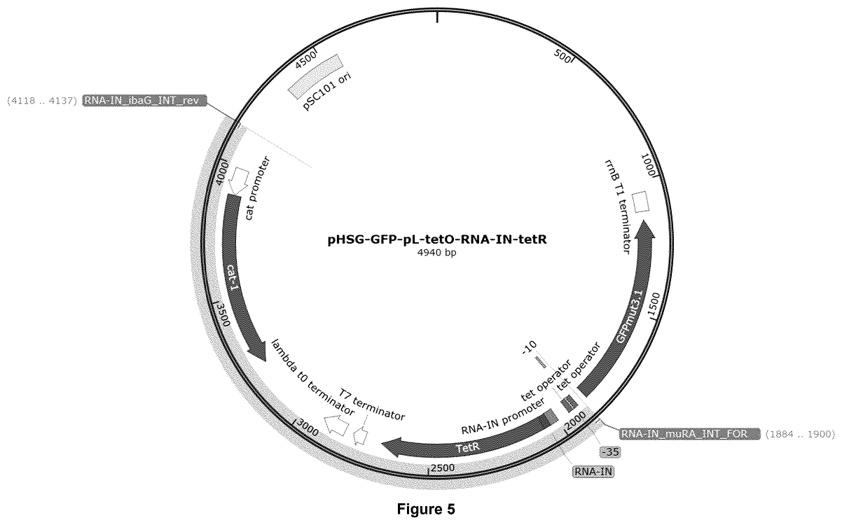

The newly cloned pHSG_GFP-pL_tetO-RNA_IN-tetR vector served as a template for a PCR using primers containing 50 bp overhangs homologous to the respective integration site in the genome of JM108. The primers RNA-IN_murA_Int_FOR (SEQ ID NO: 28) and RNA-IN_ibaG_INT_rev (SEQ ID NO: 29) were used for the amplification (represented as the grey highlighted section in FIG. 5). The PCR was carried out using the TAKARA PrimeSTAR® GXLPolymerase according to the provided instructions by TAKARA.

### 1.3 Genome Integration

The generated integration cassette was used for the genome integration by homologous recombination. For this task the target strain JM108 was transformed with pJM46, a plasmid harboring a rhamnose-inducible Red system with Gam, Bet, and Exo enzymes. After successful transformation of pJM46 a colony was picked and cultivated in LB-broth containing 2 mM Rhamnose until an OD of 0.8 was achieved. Subsequently, the suspension was washed once with 30 mL ice cold RO-water (centrifuged with 4800×g) and twice with 1 mL ice cold RO-Water (centrifuged with 12000 rpm). Afterwards, the cells were resuspended in 200 μL of RO-water and an aliquot of 50 μL was transformed with 500 ng of the double stranded, linear DNA integration cassette by electroporation (settings: 1800V, 200 Ohm, 25 μF). Cells were recovered over-night in SOC-Media containing 200 ng/ml anhydrotetracycline (aTc). After recovery, the cells were plated on LB-Agar plates containing 200 ng/ml aTc and 10 μg/mL Chloramphenicol as a selection marker for successful integration.

Formed colonies were screened by colony PCR to verify the correct integration. Therefore, the primers murA_RNA_seq_FOR (SEQ ID NO: 30) and CAT_seq_FOR (SEQ ID NO: 31) were used, which results in a fragment of 1947 bp if the integration was correct. PCR was carried out using the NEB OneTaq® Quick-Load Polymerase according to the provided instructions. 16 colonies were screened and all of them showed the correct genome modification.

### 2. Cloning of RNAOUT-Plasmid

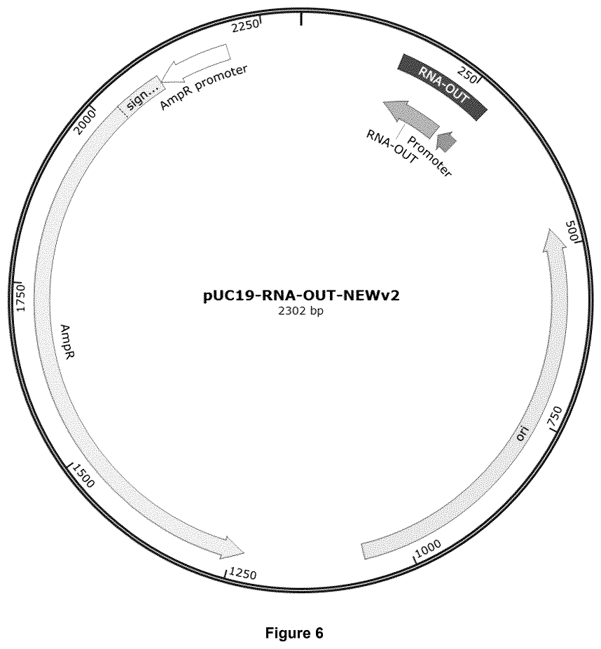

For cloning the RNAOUTv2-Plasmid, pUC19 backbone was chosen (pBR322 ORI) [GenBank: M77789.2]. Therefore, the backbone was amplified using a pUC19 plasmid as template and the Primers Bbsl_pUC_rev (SEQ ID NO: 32) and Bbsl_pUC_back (SEQ ID NO: 33). The PCR was carried out using the NEB Q5R Polymerase according to the provided instruction by NEB. The resulting PCR product (shown in FIG. 6) was used as backbone for a Golden Gate assembly using Bbsl to clone the insert gBLOCK_RNAOUTv2 into it (SEQ ID NO: 36/37).

However, also the RNA-OUT shown in SEQ ID NO: 16, 23, 24 or 25 can be used.

### 3. Proof of Selection System Based on RAIN/RNAOUT Interaction

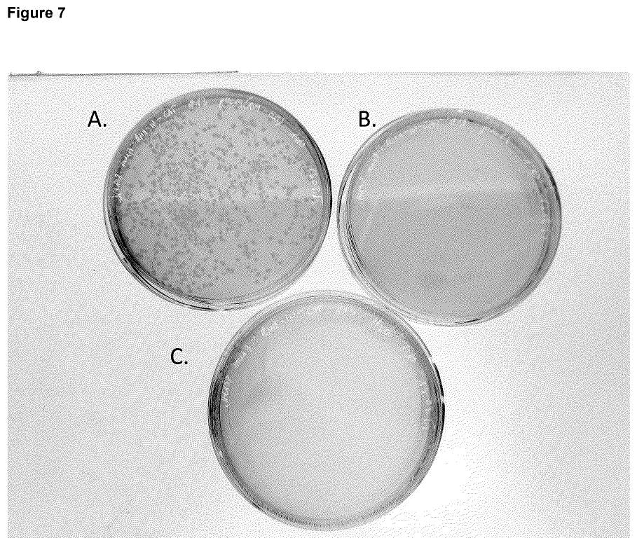

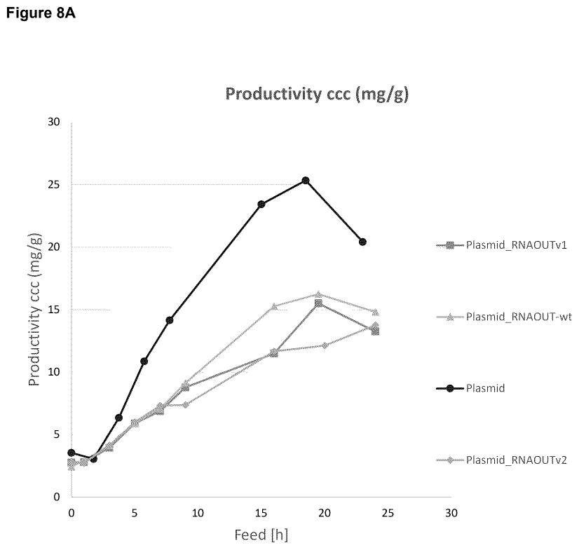

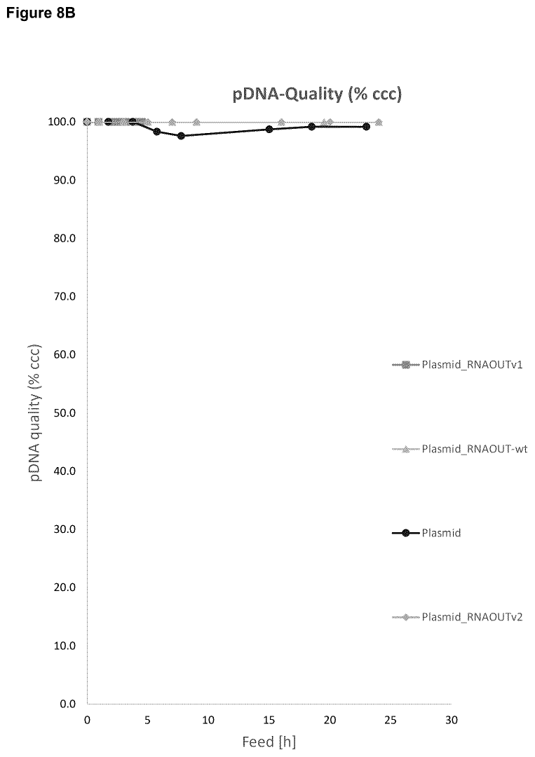

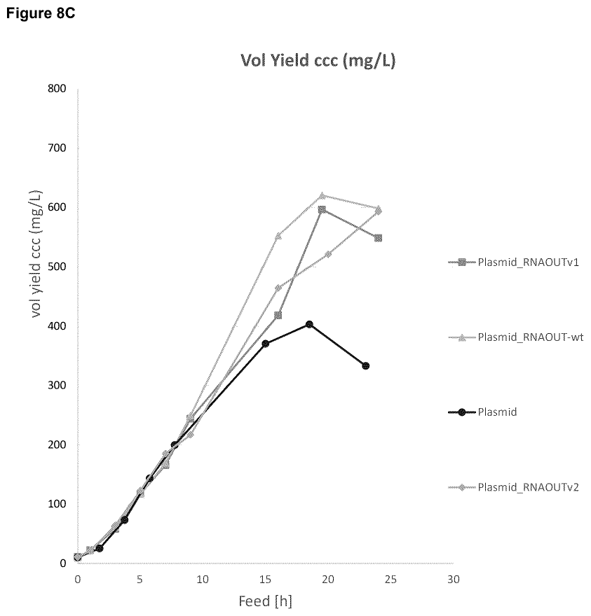

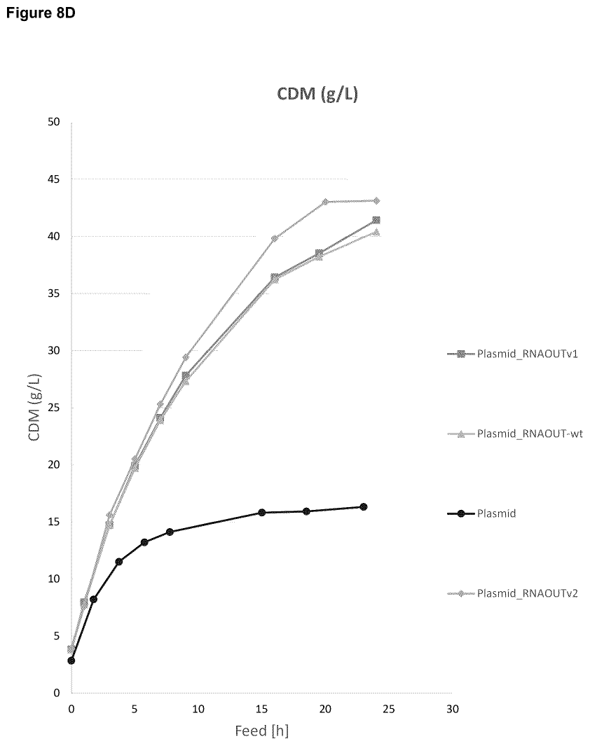

The newly constructed JM108_RAIN was transformed with the plasmid pUC19_RNAOUTv2 to check the selection based on RNA-IN/RNA-OUT interaction. Colonies only appeared on the transformation plates. The controls (JM108_RAIN+pUC19 and empty JM108) show no colonies. The result is shown in FIG. 7.

### Example 2: Plasmid Production and Quality

Bacterial host cells pursuant to the present invention were cultured under standard fed batch conditions with a linear feed profile (feeding 0,26 g/min of a 329.88 g/L glucose monohydrate solution) at 37° C., harvested by centrifugation and pDNA was isolated using alkaline lysis and pDNA yields were analyzed using HPLC analytics.

The following bacterial host cells as described herein were used:

Bacterial host cells equipped with an RNA-IN sequence (SEQ ID NO: 22) and harboring pUC19 containing the RNA-OUT sequence (SEQ ID NO: 23), named “Plasmid_RNAOUT-wt” in FIGS. 8A to 8D.

Bacterial host cells equipped with an RNA-IN sequence (SEQ ID NO: 22) and harboring pUC19 containing the modified RNA-OUT sequence (SEQ ID NO: 24), named “Plasmid_RNAOUTv1” in FIGS. 8A to 8D.

Bacterial host cells equipped with an RNA-IN sequence (SEQ ID NO: 22) and harboring pUC19 containing the modified RNA-OUT sequence (SEQ ID NO: 25), named “Plasmid_RNAOUTv2” in FIGS. 8A to 8D.

E. coli JM108 cells harboring pUC19 as a control, named “Plasmid” in Figures (A to 8D.

FIG. 8A shows that bacterial host cells as described herein produce cccDNA. It is apparent that the control (“Plasmid”) bacterial cells show a higher productivity, but as is apparent from FIG. 8C, bacterial host cells as described herein show a higher volumetric yield. This is because, they produce more cell dry mass compared to the control (see FIG. 8D) In fact, much to the surprise of the present inventor, bacterial host cells as described herein produce much more biomass than the control. This means that the presence of a plasmid containing RNA-OUT seems to enhance growth of bacterial host cells. As a result, more biomass is produced and, thus, the volumetric yield is higher (see FIG. 8C).

With regards to pDNA quality, it is apparent from FIG. 8D that bacterial host cells as described herein produce pDNA quality which is equivalent to pDNA from E. coli JM108 cells transformed with pUC19.

For pDNA product analysis, the sampling volume of the cell suspension, corresponding to 20 mg CDM, was estimated via direct measurement of the OD600. The calculated amount was transferred to 2.0 mL reaction tubes and centrifuged at 16, 100 rcf and 4 C for 10 min. The supernatant was discarded, and the cell pellets were stored at −20° C.

The content of pDNA in ccc-conformation was determined using AIEX-HPLC (CIMac™ pDNA-0.3 Analytical Column, 1.4 μμL; BIA Separations d.o.o., Slovenia). The column separated open circular, linear, and supercoiled pDNA fractions into distinct peaks. Quantification was achieved using a calibration curve based on peak areas obtained from purified pDNA samples. For HPLC analysis cell disintegration was performed by an alkaline lysis method. The obtained lysate was directly analyzed by HPLC (Agilent 1100 with a quaternary pump and diode-array detector (DAD)). Values derived from three biological replicates have a coefficient of variation lower than 10%.

1. A non-naturally occurring bacterial cell containing
(a) a nucleotide sequence
(a1) encoding at least one repressor protein which indirectly inhibits growth of said bacterial cell by repressing transcription of a gene encoding a protein which is essential for growth of said bacterial cell, the expression of which is to be regulated, and, operably linked thereto,
(a2) a nucleotide sequence encoding parts of an RNA-IN sequence,
(i) which is complementary to an RNA-OUT sequence that is transcribable from a plasmid containing said RNA-OUT sequence, and
(ii) which is present upstream of the nucleotide sequence encoding said repressor protein together with a ribosome binding site which is upstream or downstream of parts of said RNA-IN sequence,
wherein the parts of said RNA-IN sequence are designed and positioned such that they guarantee sufficient RNA-RNA interaction of the complementary sequences, so that when the plasmid with an RNA-OUT sequence is present in said bacterial cell, the RNA-OUT transcribed from said plasmid binds to RNA-IN transcribed from said nucleotide sequence of (a) in an extent sufficient to inhibit translation of the mRNA encoding said at least one repressor protein.

2. The bacterial cell of claim 1, wherein said at least one repressor protein is the Tet repressor.

3. The bacterial cell of claim 1 or 2, further containing
(b) a nucleotide sequence
(b1) encoding a protein which is essential for growth of said bacterial cell, the expression of which is to be regulated, and, operably linked thereto,
(b2) a nucleotide sequence comprising a promoter and operator which is recognized and bound by said repressor protein of (a);
wherein the parts of said RNA-IN sequence are designed and positioned such that they guarantee sufficient RNA-RNA interaction of the complementary sequences, so that when the plasmid with an RNA-OUT sequence is present in said bacterial cell, the RNA-OUT transcribed from said plasmid binds to RNA-IN transcribed from said nucleotide sequence of (a) in an extent sufficient to inhibit translation of the mRNA encoding said indirect repressor protein, thereby allowing expression of said protein of (b).

4. The bacterial cell of claim 3, wherein said protein which is essential for growth of said bacterial cell is MurA.

5. The bacterial cell of any one of the preceding claims, wherein said nucleotide sequence (a) and (b) is integrated in the genome of said bacterial cell.

6. The bacterial cell of any one of the preceding claims, wherein said nucleotide sequence (a) and (b) is a nucleotide sequence that is foreign to said bacterial cell.

7. The bacterial cell of any one of the preceding claims, wherein said nucleotide sequence (a2) is inserted between the ribosomal binding site and the start codon of said nucleotide sequence (a1).

8. The bacterial cell of any one of the preceding claims, wherein said nucleotide sequence (a1) and said nucleotide sequence (a2) are linked such that they are transcribed as one RNA.

9. The bacterial host cell of any one of the preceding claims, wherein the RNA-IN sequence is preceded by a promoter.

10. The bacterial host cell of claim 9, wherein said promoter is constitutive or inducible.

11. The bacterial cell of any one of the preceding claims, wherein said bacterial cell is transformed with said plasmid containing said RNA-OUT sequence.

12. The bacterial cell of claim 11, wherein said plasmid further contains a gene of interest.

13. The bacterial cell of claim 12, wherein said gene of interest is operably linked with a eukaryotic promoter that allows expression in a mammalian cell.

14. The bacterial cell of any one of claims 11 to 13, wherein said plasmid is a high copy number plasmid.

15. The bacterial cell of any one of claims 11 to 14, wherein said plasmid has a size of more than 5 kb.

16. The bacterial cell of any one of the preceding claims, which is an Escherichia coli cell.

17. A method for producing plasmid DNA, comprising the steps of
transforming a population of bacterial cells of any one of claims 1 to 16 with a plasmid containing an RNA-OUT sequence;
growing said population of bacterial host cell under conditions in which said repressor protein is expressible in the bacterial cells, whereby expression of said repressor protein completely or partially inhibits growth of plasmid-free bacterial cells such that plasmid-bearing bacterial cells outgrow plasmid-free cells; and
harvesting plasmid-bearing bacterial cells.

18. The method of claim 17, further comprising
isolating and purifying plasmid DNA from said harvested bacterial cell.

19. A method for producing a protein of interest, comprising the steps of
transforming a population of bacterial cells of any one of claims 1 to 16 with a plasmid containing an RNA-OUT sequence and further containing a nucleotide sequence encoding a protein of interest under the control of a prokaryotic promoter that allows expression of said protein in said bacterial host cells;
growing said population of bacterial host cell under conditions in which said repressor protein is expressible in the bacterial cells, whereby expression of said repressor protein completely or partially inhibits growth of plasmid-free bacterial cells such that plasmid-bearing bacterial cells outgrow plasmid-free cells; and
harvesting the protein of interest from plasmid-bearing bacterial cells.

20. The method of claim 19, further comprising
isolating and purifying said protein of interest.
